(19) United States
(12) Patent Application Publication
(10) Pub. No.: US 2015/0328272 A1
(43) Pub. Date: Nov. 19, 2015

(54) Compositions for Improving the Development of Arteriosclerotic Vascular Diseases

(71) Applicant: Golden Biotechnology Corporation, New Jersey, NJ (US)

(72) Inventors: Sheng-Yung LIU, New Taipei City (TW); Wu-Che WEN, New Taipei City (TW)

(21) Appl. No.: 14/410,566

(22) PCT Filed: Jun. 17, 2013
(86) PCT No.: PCT/US2013/046194

§ 371 Date: Dec. 22, 2014

Related U.S. Application Data
(60) Provisional application No. 61/663,495, filed on Jun. 22, 2012.

(51) Int. Cl. A61K 36/71; A61K 36/03; A61K 36/355; A61K 9/00

(52) U.S. Cl. CPC A61K 36/71; A61K 9/0019; A61K 9/0053; A61K 36/03; A61K 36/355

(57) ABSTRACT

The present invention provides methods and compositions for treating arteriosclerotic vascular diseases by pharmaceutical compositions comprising a component of at least one species from the genus Sargassum; a component of at least one species from the genus Lonicera; and a component of at least one species from the genus Cimicifuga.

### CROSS REFERENCE

This application claims the benefit of U.S. provisional application Ser. No. 61/663,495, filed Jun. 22, 2012, which is incorporated by reference in its entirety.

### BACKGROUND OF THE INVENTION

Atherosclerosis (also known as arteriosclerotic vascular disease or ASVD) is a condition in which an artery wall thickens as a result of the accumulation of fatty materials such as cholesterol. It is a syndrome affecting arterial blood vessels, a chronic inflammatory response in the walls of arteries, caused largely by the accumulation of macrophage white blood cells and promoted by low-density lipoproteins (plasma proteins that carry cholesterol and triglycerides) without adequate removal of fats and cholesterol from the macrophages by functional high density lipoproteins (HDL), (see apoA-1 Milano). It is commonly referred to as a hardening or furring of the arteries. It is caused by the formation of multiple plaques within the arteries. Atherosclerosis affects the entire artery tree, but mostly larger, high-pressure vessels such as the coronary, renal, femoral, cerebral, and carotid arteries.

Atherosclerotic lesions or atherosclerotic plaques are separated into two broad categories: Stable and unstable (also called vulnerable). The pathobiology of atherosclerotic lesions is very complicated but generally, stable atherosclerotic plaques, which tend to be asymptomatic, are rich in extracellular matrix and smooth muscle cells, while, unstable plaques are rich in macrophages and foam cells and the extracellular matrix separating the lesion from the arterial lumen (also known as the fibrous cap) is usually weak and prone to rupture. Ruptures of the fibrous cap, expose thrombogenic material, such as collagen to the circulation and eventually induce thrombus formation in the lumen. Upon formation, intraluminal thrombi can occlude arteries outright (i.e. coronary occlusion), but more often they detach, move into the circulation and eventually occlude smaller downstream branches causing thromboembolism (i.e. Stroke is often caused by thrombus formation in the carotid arteries). Apart from thromboembolism, chronically expanding atherosclerotic lesions can cause complete closure of the lumen. Interestingly, chronically expanding lesions are often asymptomatic until lumen stenosis is so severe that blood supply to downstream tissue(s) is insufficient resulting in ischemia.

Platelet-derived growth factor (PDGF) functions as a primary mitogen and chemoattractant for cells of mesenchymal origin. Members of the PDGF family play an important role during embryonic development and contribute to the maintenance of connective tissue in adults. Deregulation of PDGF signaling has been linked to atherosclerosis, pulmonary hypertension and organ fibrosis.

### SUMMARY OF THE INVENTION

In one aspect provides herein for the treatment of atherosclerosis comprising administering to a subject a therapeutically effective amount of a composition comprising a component of at least one species from the genus Sargassum; a component of at least one species from the genus Lonicera; and a component of at least one species from the genus Cimicifuga.

In another aspect provides herein methods of inhibiting the production or progression of one or more atherosclerotic lesions within the vasculature of a subject, comprising administering to the subject in need a therapeutically effective amount of a composition comprising a component of at least one species from the genus Sargassum; a component of at least one species from the genus Lonicera; and a component of at least one species from the genus Cimicifuga.

In another aspect provides herein methods for preventing or treating an inflammation-related arteriosclerotic vascular disease in a subject comprising administering to the subject a therapeutically effective amount of a composition comprising a component of at least one species from the genus Sargassum; a component of at least one species from the genus Lonicera; and a component of at least one species from the genus Cimicifuga.

In another aspect provides herein methods of reducing C-reactive protein in a subject comprising administering to the subject a therapeutically effective amount of a composition comprising a component of at least one species from the genus Sargassum; a component of at least one species from the genus Lonicera; and a component of at least one species from the genus Cimicifuga.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates cross-section photograph of mouse vessel (HSING-CHUN CHUNG, 2008 Dissertation, title, “Novel inhibitory effect of Antrodia camphorate on smooth muscle cell migration and carotid neointima formation in mice”).

FIG. 2A-B show illustrative results of cytotoxic effect of exemplary Composition 1 at different concentrations on smooth muscle cells (A7r5) via MTT assay (2A) and LDH assay (2B).

FIG. 3 show illustrative results of exemplary Composition 1 inhibiting PDGF-treated smooth muscle cell (A7r5) proliferation at different concentrations.

FIG. 4 provides illustrative results of 24-hour examination of PDGF-stimulated smooth muscle cell migration exposed to exemplary Composition 1 at different concentrations. * P<0.05 compared with 30 ng/ml PDGF.

FIG. 5 shows illustrative results of pathologic analysis of carotid artery in media area after treatment of exemplary Composition 1 under 400× microscope.

FIG. 6. shows illustrative results of pathologic analysis of carotid artery in neointima area after treatment of exemplary Composition 1 under 400× microscope.

FIG. 7 shows illustrative assessment of atherosclerotic lesions with the treatment of exemplary Composition 1.

FIG. 8 shows illustrative pathologic analysis of aorta in ApoE mice fed with normal diet and high-fat diet under microscope.

FIG. 9 shows illustrative assessment of serum cholesterol levels in ApoE mice with or without exemplary Composition 1 treatment.

### DETAILED DESCRIPTION OF THE INVENTION

When atherosclerosis leads to symptoms, some symptoms such as angina pectoris can be treated. Non-pharmaceutical means are usually the first method of treatment, such as cessation of smoking and practicing regular exercise. If these methods do not work, medicines are usually the next step in treating cardiovascular diseases, and, with improvements, have increasingly become the most effective method over the long term. Common medicines for atherosclerosis (or arteriosclerotic vascular disease) include a group of medications referred to as statins. They have relatively few short-term or longer-term undesirable side-effects. The invention compositions (comprising at least one species from the genus Sargassum; a component of at least one species from the genus Lonicera; and a component of at least one species from the genus Cimicifuga), in some embodiments, are obtained from extracts of natural products comprising at least one species from the genus Sargassum; a component of at least one species from the genus Lonicera; and a component of at least one species from the genus Cimicifuga and provide reduced complications and/or side effects. In some embodiments, provided herein are methods for the treatment of atherosclerosis by administering a composition comprising at least one species from the genus Sargassum; a component of at least one species from the genus Lonicera; and a component of at least one species from the genus Cimicifuga provided herein to a subject (e.g. a human). The compositions provide therapeutic benefit to a subject being treated for atherosclerosis or its related symptoms (see Examples 1-9).

In some embodiments, there are provided methods for the treatment of atherosclerosis comprising administering to a subject a therapeutically effective amount of a composition comprising a component of at least one species from the genus Sargassum; a component of at least one species from the genus Lonicera; and a component of at least one species from the genus Cimicifuga.

In some embodiments, the composition in the methods inhibits PDGF-stimulated smooth muscle cell proliferation or migration. In some embodiments, the atherosclerosis is associated with coronary artery disease, aneurysm, arteriosclerosis, myocardial infarction, embolism, stroke, thrombosis, angina, vascular plaque inflammation, vascular plaque rupture, Kawasaki disease, calcification or inflammation. In some embodiments, the subject is human. See Examples 2-9.

In some embodiments, the composition (comprising a component of at least one species from the genus Sargassum; a component of at least one species from the genus Lonicera; and a component of at least one species from the genus Cimicifuga) is prepared by any means that can obtain a therapeutically effective amount of the composition. For example, the components are prepared from any parts of the plants; in dry or wet forms; by extraction in liquid or solid from; with or without freeze-drying. In some embodiments, the invention compositions are prepared by extraction of a component or components from each of the at least one species from the genus Sargassum, Lonicera, and Cimicifuga.

In some embodiments, the composition inhibits PDGF-stimulated smooth muscle cell proliferation or migration. In some embodiments, the composition reduced neointima formation. In some embodiments, the composition inhibits the production or progression of one or more atherosclerotic lesions within the vasculature of a subject. In some embodiments, the composition prevents or treats an inflammation-related arteriosclerotic vascular disease in a subject. In some embodiments, the composition is administered by injection. In some embodiments, the composition is administered orally. In certain embodiments, the subject is human.

The non-limited exemplary compositions are illustrated below. For example, Composition 1 is prepared from aqueous extraction of at least one species from the genus Sargassum (e.g., Sargassum siliquastrum Ag), at least one species from the genus Lonicera (e.g., Lonicera japonica Thunb), and at least one species from the genus Cimicifuga (e.g., Cimicifuga foetida, L. var. intermedia Regel). In some embodiments, the aqueous solvents may be heated. In some embodiments, the aqueous solvents may be acidic. In some embodiments, the aqueous solvents may be basic. In some embodiments, the aqueous solvents may be neutral. For example, exemplary Composition 1 is isolated from aqueous solvent extracts. In certain embodiments, the aqueous solvent is water. In certain embodiments, the aqueous solvent is heated.

In other embodiments, the invention compositions are prepared from the organic solvent extractions of at least one species from the genus Sargassum (e.g., Sargassum siliquastrum Ag), at least one species from the genus Lonicera (e.g., Lonicera japonica Thunb), and at least one species from the genus Cimicifuga (e.g., Cimicifuga foetida, L. var. intermedia Regel). In some embodiments, the organic solvent is selected from alcohols (e.g., methanol, ethanol, propanol, or the like), esters (e.g., methyl acetate, ethyl acetate, or the like), alkanes (e.g., pentane, hexane, heptane, or the like), halogenated alkanes (e.g., chloromethane, chloroethane, chloroform, methylene chloride, and the like), and the like. For example, exemplary Composition 1 is isolated from organic solvent extracts. In certain embodiments, the organic solvent is alcohol. In certain embodiments, the alcohol is ethanol.

In some embodiments, the composition comprises about 1% to about 99% of at least one species from the genus Sargassum by weight, about 1% to about 99% of at least one species from the genus Lonicera by weight, and about 1% to about 99% of at least one species from the genus Cimicifuga by weight by weight.

In some embodiments, the vasculature comprises a cardiac artery. In certain embodiments, the vasculature comprises an aorta. In some embodiments, the subject is human.

In some embodiments, the compositions provided herein possess the therapeutic effects of inhibiting the production or progression of atherosclerotic lesions. See Example 8.

In some embodiments provide methods for preventing or treating an inflammation-related arteriosclerotic vascular disease in a subject comprising administering to the subject a therapeutically effective amount of a composition comprising a component of at least one species from the genus Sargassum; a component of at least one species from the genus Lonicera; and a component of at least one species from the genus Cimicifuga.

In some embodiments provide methods reducing C-reactive protein in a subject comprising administering to the subject a therapeutically effective amount of a composition comprising a component of at least one species from the genus Sargassum; a component of at least one species from the genus Lonicera; and a component of at least one species from the genus Cimicifuga.

### Certain Pharmaceutical and Medical Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

The term “acceptable” with respect to a formulation, composition or ingredient, as used herein, means having no persistent detrimental effect on the general health of the subject being treated.

Sargassum is a genus of brown (class Phaeophyceae) macroalga (seaweed) in the order Fucales. Numerous species are distributed throughout the temperate and tropical oceans of the world, where they generally inhabit shallow water and coral reefs. However, the genus may be best known for its planktonic (free-floating) species. Some of the species in this genus (e.g., Sargassum siliquastrum Ag) are have medicinal properties, and have been used in Taiwan as a traditional medicine. In some embodiments, the Sargassum species is selected from the group consisting of Sargassum siliquastrum Ag, Sargassum pallidum Ag, Sargassum fusiforme Setch, and the like.

Lonicera especially Lonicera japonica (as known as Japanese Honeysuckle, Suikazura in Japanese; Jinyinhua in Chinese) is a species of honeysuckle native to eastern Asia including China (northern and eastern P.R. China and Taiwan), Japan, and Korea. The Japanese Honeysuckle flower is of high medicinal value in traditional Chinese medicine; it is thought to have antibacterial and anti-inflammatory properties. Traditional indications for use of this formula include fever, headache, cough, thirst, and sore throat. In some embodiments, the Lonicera species is selected from the group consisting of Lonicera japonica Thunb, Lonicera periclymenum, and Lonicera sempervirens.

Cimicifuga (bugbane or cohosh) is a genus of between 12-18 species of flowering plants belonging to the family Ranunculaceae, native to temperate regions of the Northern Hemisphere. Cimicifuga, especially Cimicifuga foetida, L. var. intermedia Regel (Rhizoma Cimicifugae), is pungent and sweet in flavor, slightly cold in nature and acting on the lung, spleen and stomach channels. In some embodiments, the Cimicifuga species is selected from the group consisting of Cimicifuga foetida, L. var. intermedia Regel, Cimicifuga simplex, Cimicifuga heracleifolia, Kom, Cimicifuga dahurica (Turcz.) Maxim and Cimicifuga racemosa (L.) Nutt.

The term “carrier,” as used herein, refers to relatively nontoxic chemical compositions or agents that facilitate the incorporation of an invention composition into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compositions that are used to dilute the invention composition of interest prior to delivery. Diluents can also be used to stabilize compositions because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a composition provided herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising an invention composition as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. an invention composition (i.e., a composition comprising a component of at least one species from the genus Sargassum; a component of at least one species from the genus Lonicera; and a component of at least one species from the genus Cimicifuga described herein) and a co-agent, are both administered to a patient simultaneously in the form of a single entity or dosage. The term “non-fixed combination” means that the active ingredients, e.g. an invention composition (i.e., a composition comprising a component of at least one species from the genus Sargassum; a component of at least one species from the genus Lonicera; and a component of at least one species from the genus Cimicifuga described herein) and a co-agent, are administered to a patient as separate entities either simultaneously, concurrently or sequentially with no specific intervening time limits, wherein such administration provides effective levels of the two compositions in the body of the patient. The latter also applies to cocktail therapy, e.g. the administration of three or more active ingredients.

The term “a component of at least one species from the genus Sargassum” refers to any parts or components of the plant parts such as wet or dry parts, extracts, freeze-drying products, or the like.

The term “a component of at least one species from the genus Lonicera” refers to any parts or components of the plant parts such as wet or dry parts, extracts, freeze-drying products, or the like.

The term “a component of at least one species from the genus Cimicifuga” refers to any parts or components of the plant parts such as wet or dry parts, extracts, freeze-drying products, or the like.

The term “pharmaceutical composition” refers to a mixture of an exemplary invention composition (i.e., a composition comprising a component of at least one species from the genus Sargassum; a component of at least one species from the genus Lonicera; and a component of at least one species from the genus Cimicifuga described herein) with other chemical components, such as carriers, stabilizers, diluents, dispersing agents, suspending agents, thickening agents, and/or excipients. The pharmaceutical composition facilitates administration of the exemplary invention composition to an organism. Multiple techniques of administering the exemplary invention composition exist in the art including, but not limited to: intravenous, oral, aerosol, parenteral, ophthalmic, pulmonary and topical administration.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, an exemplary invention composition as described herein is administered in a local rather than systemic manner, for example, via injection of the invention composition directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the exemplary invention composition as described herein is provided in the form of a rapid release formulation, in the form of an extended release formulation, or in the form of an intermediate release formulation. In yet other embodiments, the exemplary invention composition described herein is administered topically.

In some embodiments, the exemplary invention composition is administered parenterally or intravenously. In other embodiments, the exemplary invention composition is administered by injection. In some embodiments, the exemplary invention composition is administered orally.

### Pharmaceutical Composition/Formulation

In some embodiments provide compositions comprising a component of at least one species from the genus Sargassum; a component of at least one species from the genus Lonicera; and a component of at least one species from the genus Cimicifuga.

For example, Composition 1 comprises at least three herbal components, at least one species from the genus Sargassum (e.g., Sargassum siliquastrum Ag), at least one species from the genus Lonicera (e.g., Lonicera japonica Thunb.) and at least one species from the genus Cimicifuga (Cimicifuga foetida, L. var. intermedia Regel). In some embodiments, Composition 1 comprises components of Sargassum siliquastrum Ag, Lonicera japonica Thunb, and Cimicifuga foetida, L. var. intermedia Regel.

In some embodiments provide pharmaceutical compositions comprising a therapeutically effective amount of a composition comprising a component of at least one species from the genus Sargassum; a component of at least one species from the genus Lonicera; and a component of at least one species from the genus Cimicifuga.

In some embodiments, the compositions described herein are formulated into pharmaceutical compositions. In specific embodiments, pharmaceutical compositions are formulated in a conventional manner using one or more physiologically acceptable carriers comprising excipients and auxiliaries which facilitate processing of the active compositions into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). Provided herein are pharmaceutical compositions comprising an exemplary invention composition (i.e., a composition comprising a component of at least one species from the genus Sargassum; a component of at least one species from the genus Lonicera; and a component of at least one species from the genus Cimicifuga described herein) and a pharmaceutically acceptable diluent(s), excipient(s), or carrier(s). In certain embodiments, the compositions described are administered as pharmaceutical compositions in which the exemplary invention composition (i.e., a composition comprising a component of at least one species from the genus Sargassum; a component of at least one species from the genus Lonicera; and a component of at least one species from the genus Cimicifuga described herein) is mixed with other active ingredients, as in combination therapy. Encompassed herein are all combinations of actives set forth in the combination therapies section below and throughout this disclosure. In specific embodiments, the pharmaceutical compositions include one or more compositions (i.e., a composition comprising a component of at least one species from the genus Sargassum; a component of at least one species from the genus Lonicera; and a component of at least one species from the genus Cimicifuga described herein).

A pharmaceutical composition, as used herein, refers to a mixture of an exemplary invention composition (i.e., a composition comprising a component of at least one species from the genus Sargassum; a component of at least one species from the genus Lonicera; and a component of at least one species from the genus Cimicifuga described herein) with other chemical components, such as carriers, stabilizers, diluents, dispersing agents, suspending agents, thickening agents, and/or excipients. In certain embodiments, the pharmaceutical composition facilitates administration of the exemplary invention composition to an organism. In some embodiments, practicing the methods of treatment or use provided herein, therapeutically effective amounts of compositions (i.e., a composition comprising a component of at least one species from the genus Sargassum; a component of at least one species from the genus Lonicera; and a component of at least one species from the genus Cimicifuga described herein) are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the exemplary invention composition used and other factors. The compositions described herein are used singly or in combination with one or more therapeutic agents as components of mixtures.

In one embodiment, an exemplary invention composition (i.e., a composition comprising a component of at least one species from the genus Sargassum; a component of at least one species from the genus Lonicera; and a component of at least one species from the genus Cimicifuga described herein) is formulated in an aqueous solution. In specific embodiments, the aqueous solution is selected from, by way of example only, a physiologically compatible buffer, such as Hank's solution, Ringer's solution, or physiological saline buffer. In other embodiments, an exemplary invention composition (i.e., a composition comprising a component of at least one species from the genus Sargassum; a component of at least one species from the genus Lonicera; and a component of at least one species from the genus Cimicifuga described herein) is formulated for transmucosal administration. In specific embodiments, transmucosal formulations include penetrants that are appropriate to the barrier to be permeated. In still other embodiments wherein the compositions described herein are formulated for other parenteral injections, appropriate formulations include aqueous or nonaqueous solutions. In specific embodiments, such solutions include physiologically compatible buffers and/or excipients.

In another embodiment, compositions described herein are formulated for oral administration. Compositions described herein, including an exemplary invention composition (i.e., a composition comprising a component of at least one species from the genus Sargassum; a component of at least one species from the genus Lonicera; and a component of at least one species from the genus Cimicifuga described herein), are formulated by combining the active compositions with, e.g., pharmaceutically acceptable carriers or excipients. In various embodiments, the compositions described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipients with one or more of the compositions described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active exemplary invention composition doses.

In certain embodiments, therapeutically effective amounts of at least one of the compositions described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain the exemplary invention composition that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compositions described herein are formulated for buccal or sublingual administration. Formulations suitable for buccal or sublingual administration include, by way of example only, tablets, lozenges, or gels. In still other embodiments, the compositions described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions of the exemplary invention composition (i.e., a composition comprising a component of at least one species from the genus Sargassum; a component of at least one species from the genus Lonicera; and a component of at least one species from the genus Cimicifuga described herein) are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compositions in water-soluble form. In additional embodiments, suspensions of the active compositions are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compositions to allow for the preparation of highly concentrated solutions. Alternatively, in other embodiments, the active ingredient is in powder form for constitution with a suitable vehicle, e.g., sterile pyrogen-free water, before use.

In one aspect, compositions (i.e., compositions described herein) are prepared as solutions for parenteral injection as described herein or known in the art and administered with an automatic injector. Automatic injectors, such as those disclosed in U.S. Pat. Nos. 4,031,893, 5,358,489; 5,540,664; 5,665,071, 5,695,472 and WO/2005/087297 (each of which are incorporated herein by reference for such disclosure) are known. In general, all automatic injectors contain a volume of solution that includes the exemplary invention composition (i.e., a composition comprising a component of at least one species from the genus Sargassum; a component of at least one species from the genus Lonicera; and a component of at least one species from the genus Cimicifuga described herein) to be injected. In general, automatic injectors include a reservoir for holding the solution, which is in fluid communication with a needle for delivering the drug, as well as a mechanism for automatically deploying the needle, inserting the needle into the patient and delivering the dose into the patient. Exemplary injectors provide about 0.3 mL, 0.6 mL, 1.0 mL or other suitable volume of solution at about a concentration of 0.5 mg to 50 mg of the exemplary invention composition (i.e., a composition comprising a component of at least one species from the genus Sargassum; a component of at least one species from the genus Lonicera; and a component of at least one species from the genus Cimicifuga described herein) per 1 mL of solution. Each injector is capable of delivering only one dose of the exemplary invention composition.

In still other embodiments, the compositions (i.e., compositions described herein) are administered topically. The compositions described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compositions (i.e., compositions described herein) are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the exemplary invention composition (i.e., a composition comprising a component of at least one species from the genus Sargassum; a component of at least one species from the genus Lonicera; and a component of at least one species from the genus Cimicifuga described herein) is accomplished by means of iontophoretic patches and the like. In certain embodiments, transdermal patches provide controlled delivery of the exemplary invention composition (i.e., a composition comprising a component of at least one species from the genus Sargassum; a component of at least one species from the genus Lonicera; and a component of at least one species from the genus Cimicifuga described herein). In specific embodiments, the rate of absorption is slowed by using rate-controlling membranes or by trapping the exemplary invention composition within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the exemplary invention composition optionally with carriers, optionally a rate controlling barrier to deliver the exemplary invention composition to the skin of the host at a controlled and predetermined rate over a prolonged period of time, and means to secure the device to the skin.

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal dosage forms described herein may incorporate certain pharmaceutically acceptable excipients which are conventional in the art. In one embodiment, the transdermal formulations described herein include at least three components: (1) a formulation of the exemplary invention composition (i.e., a composition comprising a component of at least one species from the genus Sargassum; a component of at least one species from the genus Lonicera; and a component of at least one species from the genus Cimicifuga described herein); (2) a penetration enhancer; and (3) an aqueous adjuvant. In addition, transdermal formulations can include additional components such as, but not limited to, gelling agents, creams and ointment bases, and the like. In some embodiments, the transdermal formulations further include a woven or non-woven backing material to enhance absorption and prevent the removal of the transdermal formulation from the skin. In other embodiments, the transdermal formulations described herein maintain a saturated or supersaturated state to promote diffusion into the skin.

In other embodiments, the compositions (i.e., compositions described herein) are formulated for administration by inhalation. Various forms suitable for administration by inhalation include, but are not limited to, aerosols, mists or powders. Pharmaceutical compositions of the exemplary invention composition (i.e., a composition comprising a component of at least one species from the genus Sargassum; a component of at least one species from the genus Lonicera; and a component of at least one species from the genus Cimicifuga described herein) are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatins for use in an inhaler or insufflator are formulated containing a powder mix of the exemplary invention composition and a suitable powder base such as lactose or starch.

Intranasal formulations are known in the art and are described in, for example, U.S. Pat. Nos. 4,476,116, 5,116,817 and 6,391,452, each of which is specifically incorporated herein by reference. Formulations, which include the exemplary invention composition (i.e., a composition comprising a component of at least one species from the genus Sargassum; a component of at least one species from the genus Lonicera; and a component of at least one species from the genus Cimicifuga described herein), which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are found in sources such as REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compositions described herein, may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the exemplary invention composition described herein and a suitable powder base such as lactose or starch.

In still other embodiments, the compositions (i.e., compositions described herein) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compositions into preparations which can be used pharmaceutically. Proper formulation is dependent upon the route of administration chosen. Any pharmaceutically acceptable techniques, carriers, and excipients are optionally used as suitable and as understood in the art. Pharmaceutical compositions comprising an exemplary invention composition (i.e., a composition comprising a component of at least one species from the genus Sargassum; a component of at least one species from the genus Lonicera; and a component of at least one species from the genus Cimicifuga described herein) may be manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one exemplary invention composition (i.e., herbal compositions described herein) described herein as an active ingredient. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the exemplary invention composition described herein include formulating the invention compositions with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which an exemplary invention composition is dissolved, emulsions comprising the exemplary invention composition, or a solution containing liposomes, micelles, or nanoparticles comprising the exemplary invention composition as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising at least the exemplary invention composition (i.e., invention herbal compositions described herein) illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, pharmaceutical aqueous suspensions include one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally include solubilizing agents to aid in the solubility of an exemplary invention composition (i.e., herbal compositions described herein). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compositions such as benzalkonium chloride, cetyltrimethylammonium bromide and cetylpyridinium chloride.

Still other pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions may include one or more antioxidants to enhance chemical stability where required. Suitable antioxidants include, by way of example only, ascorbic acid and sodium metabisulfite.

In certain embodiments, pharmaceutical aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compositions are employed. Liposomes and emulsions are examples of delivery vehicles or carriers herein. In certain embodiments, organic solvents such as N-methylpyrrolidone are also employed. In additional embodiments, the compositions described herein are delivered using a sustained-release system, such as semipermeable matrices of solid hydrophobic polymers containing the therapeutic agent. Various sustained-release materials are useful herein. In some embodiments, sustained-release capsules release the compositions for a few hours up to over 24 hours. Depending on the chemical nature and the biological stability of the therapeutic reagent, additional strategies for protein stabilization may be employed.

In certain embodiments, the formulations described herein include one or more antioxidants, metal chelating agents, thiol containing compositions and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Combination Treatments

In general, the compositions described herein and, in embodiments where combinational therapy is employed, other agents do not have to be administered in the same pharmaceutical composition, and in some embodiments, because of different physical and chemical characteristics, are administered by different routes. In some embodiments, the initial administration is made according to established protocols, and then, based upon the observed effects, the dosage, modes of administration and times of administration is modified by the skilled clinician.

In some embodiments, therapeutically-effective dosages vary when the drugs are used in treatment combinations. Combination treatment further includes periodic treatments that start and stop at various times to assist with the clinical management of the patient. For combination therapies described herein, dosages of the co-administered compositions vary depending on the type of co-drug employed, on the specific drug employed, on the disease, disorder, or condition being treated and so forth.

It is understood that in some embodiments, the dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in other embodiments, the dosage regimen actually employed varies widely and therefore deviates from the dosage regimens set forth herein.

Combinations of compositions (i.e., the composition comprising a component of at least one species from the genus Sargassum; a component of at least one species from the genus Lonicera; and a component of at least one species from the genus Cimicifuga described herein) with other suitable agents for the treatment of atherosclerosis are intended to be covered. In some embodiments, examples of suitable agents for the treatment of atherosclerosis include, but are not limited to, the following: statins such as atorvastatin, fluvastatin, lovastatin, pitavastatin, pravastatin, rosuvastatin, simvastatin, combinations thereof, or the like; photosensitizers such as Motexafin lutetium; MK-0524A (niacin ER and laropiprant); anti-oxidants such as AC3056; anti-inflammatory agents such as steroids, non-steroidal anti-inflammatory drugs such as aspirin, ibuprofen, and naproxen or other COX-2 inhibitors, and the like; ACAT inhibitors such as Pactimibe, and the like; or any derivative related agent of the foregoing.

The combinations of the compositions and other suitable agents for the treatment of atherosclerosis described herein encompass additional therapies and treatment regimens with other agents in some embodiments. Such additional therapies and treatment regimens can include another agents for the treatment of atherosclerosis in some embodiments. Alternatively, in other embodiments, additional therapies and treatment regimens include other agents used to treat adjunct conditions associated with the atherosclerosis or a side effect from such agent in the combination therapy. In further embodiments, adjuvants or enhancers are administered with a combination therapy described herein.

In some embodiments provide compositions for the treatment of atherosclerosis comprising a therapeutically effective amount of a composition comprising a component of at least one species from the genus Sargassum; a component of at least one species from the genus Lonicera; and a component of at least one species from the genus Cimicifuga; and one or more statins.

### EXAMPLES

### Example 1

### Preparation of the Exemplary Compositions

One hundred grams of vegetative parts of the plants (e.g., Sargassum siliquastrum Ag, Lonicera japonica Thunb, and Cimicifuga foetida, L. var. intermedia Regel) were placed into a flask. A proper amount of water and alcohol (70-100% alcohol solution) was added into the flask and were stirred at 20-25° C. for at least 1 hour. The solution was filtered through a filter and 0.45 μm membrane and the filtrate was collected as the extract. The extract was used for further testing. Exemplary Composition 1 was prepared from this method.

Furthermore, invention compositions (including exemplary Composition 1) may also be prepare by the following procedure, or the like. Vegetative parts of the plants (e.g., Sargassum siliquastrum Ag, Lonicera japonica Thunb, and Cimicifuga foetida, L. var. intermedia Regel) are collected, cleaned, washed and cut into small pieces and oven dried at 40° C. overnight. The dried material is ground using a blender and extracted three times with hot and cold alcohol (1:10 v/v) and three times with hot and cold water or with mixtures of chloroform and alcohol. Other solvents such as acetone may be used as a medium for the extraction. This is a process designed to separate soluble components by diffusion from a solid matrix (plant tissue) using a liquid matrix (solvent). Alcohol, water, chloroform and acetone have produced good yield in extracting the active components. The extraction is done a few times. The pooled extracts are vacuum-dried at 40° C. and stored until used.

Other herbal extraction or harvesting methods known are adapted to prepare the exemplary compositions.

### Example 2

### Rat Smooth Muscle Cell Model Materials and methods

A7r5 cell line (rat aortic smooth muscle cells) was purchased from Bioresource Collection and Research Center, (Taiwan).

| | |
|---|---|
| Cell line | A7r5 (BCRC 60082) |
| Species | Rattus norvegicus |
| Morphology | Fibroblast |
| Description | Muscle; smooth; thoracic aorta |
| Growth Character | Adherent |
| Cell cultures | DMEM with 4 mM L-glutamine, 1.5 g/L |
| | sodium bicarbonate, 4.5 g/L glucose + |
| | 10% FBS |
| Cell Culture | 37° C. 5% CO2 |
| Conditions | |

### 2.1 MTT Assay

MTT assay is commonly used to determine cell proliferation, percent of viable cells, and cytotoxicity. MTT (3-[4,5-dimethylthiazol-2-yl]2,5-diphenyltetrazolium bromide) is a yellow dye, which can be absorbed by the living cells and be reduced to purplish blue formazan crystals by succinate tetrazolium reductase in mitochondria. Formazan formation can therefore be used to assess and determine the survival rate of cells. A solubilization solution (usually either dimethyl sulfoxide, an acidified ethanol solution, or a solution of the detergent sodium dodecyl sulfate in diluted hydrochloric acid) is added to dissolve the insoluble purple formazan product into a colored solution. The absorbance of this colored solution can be quantified by measuring at a certain wavelength (usually between 500 and 600 nm) by a spectrophotometer. The more surviving cells, the higher the absorbance.

The percentage of cell survival (%)=OD value of experimental group÷OD value of control group×100%.

### Procedure

1. Adherence of cells: 2×10⁴ cells/ml/well of A7r5 cells were seeded onto a 24-well plate and incubated at 37° C. for 24 hours.

2. Dosing: 500 ul/well different concentrations of Composition 1 were pretreated in culture medium containing 1% FBS/DMEM for 20 hours. The DMEM was removed and PDGF in 1% FBS/DMEM was added and incubated at 37° C. for 24 hours.

3. MTT assay: Subsequently, in the dark environment, to each well of the plates were added 50 ul/well of 5 mg/ml MTT and reacted for 3 hours. Each reaction mixture was added 500 ul/well DMSO and vibrated for 5 minute. The survival rate of cells was calculated based on the measurement of absorption at the 570 nm wavelength by ELISA reader.

### 2.2 Lactate Dehydrogenase (LDH) Activity Assay

Cells have abundant lactate dehydrogenase (LDH). When cells are healthy, LDH cannot freely cross cell membrane. However, LDH is released into the surrounding medium following loss of membrane integrity resulting from either apoptosis or necrosis where the cells exhibit rapid swelling and cease their physiological mechanisms. LDH activity in the culture medium is directly proportional to the number of dead cells. The cell viability can be measured quantitatively to detect absorbance by using colorimetric method at a wavelength of 492 nm. The change of the absorbance values come from the fact that LDH catalyzes the conversion of lactate to pyruvate with the concomitant production of NADH. The NADH, in the presence of diaphorase and tetrazolium salt INT, is used to drive the diaphorase-catalyzed production of red formazan product. The present experiment utilizes Cytotoxicity Assay Kit (Promega) to conduct culture medium LDH Quantitation assay.

### Procedure

1. Adherence of cells: 2×10⁴ cells/ml/well of A7r5 cells were seeded onto a 24-well plate and incubated at 37° C. for 24 hours.

2. Dosing: 500 ul/well different concentrations of Composition 1 were formulated in culture medium containing 10% FBS/DMEM and incubated for 24 hours. The culture medium of each well was centrifuged for 5 minutes at 400×g and the supernatants (50 μl) were transferred into another 96-well plate.

3. LDH assay: 50 μl of substrate mixed solution was added and reacted at room temperature for 30 mins in the dark. 50 μl of Stop solution was added to terminate the reaction. Absorbance was measured by ELISA reader at the 490 nm wavelength.

### 2.3 Wound Scratching Test

### Procedure

1. A7r5 cells (5×10⁶ cells/ml) were seeded onto a 6-well cell culture plate and incubated at 37° C. overnight.

2. 1×PBS was used to wash the wells twice. Composition 1 with different concentrations in DMEM culture medium containing 1% FBS was added and pretreated for 20 hours.

3. A cross-shape acellular space was created by a sterile 200 μl pipette tip and washed twice with 1×PBS.

4. After removing PBS, 2 ml PDGF in DMEM culture medium containing 1% FBS were added. The cells were photographed by a microscope at 0, 6, 12 and 24 hours, respectively from the time of adding PDGF.

### Example 3

### The Rat Atherosclerosis Model

### 3.1 Carotid Artery Ligation Model

Arteries are vessels that carry blood away from the heart. The carotid arteries are blood vessels that supply blood to the head, neck and brain. One carotid artery is position on each side of the neck. The right common carotid artery branches from the brachiocephalic artery and extends up the right side of the neck. The left common carotid artery branches from the aorta and extends up the left side of the neck. Each carotid artery branches into internal and external vessels near the top of the thyroid. Adapting a model as described by Hsing-Chun Chung (Dissertation, 2008, Southern Taiwan University), the carotid artery ligation was conducted on the left common carotid artery in mice to induce neointimal thickening.

This experiment used 8-week-old C57BL/6J male mice having about 25 g of body weight, which were purchased from National Laboratory Animal Center. These mice were maintained at the Laboratory Animal center of National Defense Medical Center on a 12 hour dark/12 hour light cycle in air conditional rooms (18-26° C., 30%-70% humidity).

1. The animals were given Composition 1 by oral gavage three days prior to the surgery and were continuously fed with Composition 1 for 28 days by oral gavage.

2. 8-week-old (C57BL/6J (B6) male mice were anesthetized with pentobarbital (50 mg/kg body weight). The left common carotid artery was ligated twice by a no. 6 silk suture at the site just proximal to the carotid bifurcation.

3. The animals were given Composition 1 after sutured. 8-10 mice of each group were sacrificed. Samples from carotid artery tissue and blood were collected and stored properly until further analysis, which included the comparative analysis of the treatment group and the control group.

### 3.2 Rat Atherosclerosis Model-ApoE Knock-Out Mice

Apo KO mice were purchased from Jackson Laboratory and maintained at National Laboratory Animal Center. The experiment was performed at Laboratory Animal center of National Defense Medical Center. 8-week-old ApoE KO mice were given preventive medication treatment three days prior to being fed with OpenSource diet (40% fat, 0.5% cholesterol) and continuously fed by oral gavage until sacrifice. During the period of the experiment, blood serums were collected from cheeks and the levels of cholesterol, C reactive protein (CRP) and ROS content in blood serums were measured.

### Example 4

### Serum Cholesterol Measurement by Cholesterol Assay Kit Preparations of Standardized Cholesterol Sample

| | 200 uM Cholesterol | Assay buffer | Final Conc. |
|---|---|---|---|
| No. | standard (ul) | (ul) | (uM) |
| 1 | 0 | 1000 | 0 |
| 2 | 10 | 990 | 2 |
| 3 | 20 | 980 | 4 |
| 4 | 30 | 970 | 6 |
| 5 | 40 | 960 | 8 |
| 6 | 60 | 940 | 12 |
| 7 | 80 | 920 | 16 |
| 8 | 100 | 990 | 20 |

### Procedure

1. Added 50 μl diluted cholesterol standard or 50 μl appropriately diluted serum.

2. Added 50 μl freshly prepared Assay Cocktail:
a. 4745 μl assay buffer
b. 150 μl cholesterol detector
c. 50 μl HRP
d. 50 μl cholesterol oxidase
e. 5 μl cholesterol esterase

3. Incubated at 37° C. for 30 minutes in the dark

4. Measure fluorescence by fluorescence detector (Excitation: OD 530-580 nm; Emission: 585-595 nm)

### Example 5

### C Reactive Protein Analysis by Enzyme-Linked Immunosorbent Assay (ELISA)

First, 200 μl/well of blocking buffer were added into 96-well ELISA plate and incubated for 1 hour at room temperature. 100 μl/well of diluted serum samples were added and incubated for 2 hours at room temperature. Then 100 μl/well of detection antibody were added and incubated for 1 hour at room temperature. Upon the completion of each incubation step mention above, the wells were washed 6 times with 400 μl/well of 0.05 PBS-T (wash buffer). Last, 100 μl/well of tetramethylbenzidine (TMB) were added and incubated for 15 minutes in the dark, and 50 μl of Stop solution were added to terminate the incubation. The absorbance of each well was read at 450 nm by ELISA reader.

### Example 6

### Histomorphology

Tissues dissected from live animals were immediately fixed in 10% formalin solution for about 24 hours, followed by dehydration using an automated tissue processor (Tissue-processor, Japan). Samples were embedded with completely melted paraffin performed by dispersing console (Tissue-Tek, USA). Then the samples were chilled for 15 minutes at 4° C. to solidify. The paraffin blocks were sectioned into single cell layers in 5 μm thickness. The paraffin sections were placed in warm water bath and the paraffin sections were fished out and plated on glass slides. The slides were baked in oven at 75° C. for 30 minutes to melt paraffin. To deparaffinise, the slides were placed in xylene for 10 minutes and then immersed in 100% ethanol for 10 minutes. The rehydration steps were performed by subsequently placing the slides for 10 seconds in 95%, 85%, and 70% ethanol, followed by rinsing in running water for 5 minutes. The slices were immersed in hematoxylin solution (Surgipath Co., USA) for 2 minutes, washed with running water for 1 minute, and then immersed in acidic alcohol (1 ml concentrated HCl in 1 L 70% ethanol) for 1 second. The slides were dipped into ammonia solution for 1 second, and then washed by water for 10 minutes. The slides were incubated in Eosin solution for 90 seconds, dehydrated through 70%, 80%, 90% and 100% ethanol, and then air-dried. The slides were mounted using histological mounting media (Histomount Co. USA). The medial and neointimal thickening in the ligation-injured mouse carotid artery was examined by optical microscope.

### Example 7

### Evaluation of Blood Vessels

### Materials

The following materials were used.
1. Olympus inverted phase contrast microscope
2. CDF 480 imaging capture system
3. Meta Imaging series 5.0

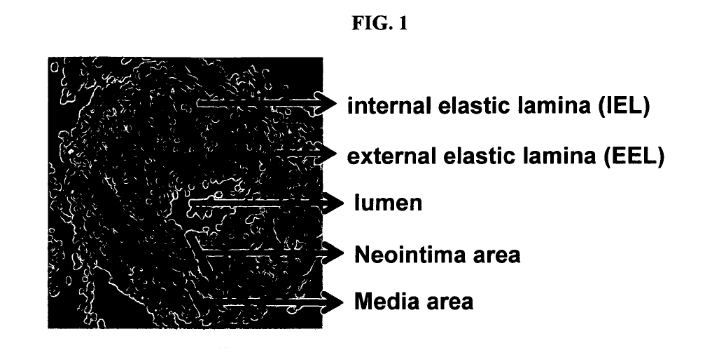

Measurement of mouse blood vessel areas after ligation is shown in FIG. 1. EEL=external elastic lamina; IEL=internal elastic lamina; Medial area=area defined by EEL−area defined by IEL; Neointima area=area defined by IEL−Lumen area; N/M ratio=neointima area/medial area.

### Example 8

### Data Assessment and Statistical Analysis

Experimental data were presented as means±S.E. N represents the numbers of animal for each group. The data were analyzed by Kruskal-Wallis test. Multi-factorial and multi-group data were analyzed by ANOVA. All statistical analysis uses SPSS 12.0 (SPSS Inc. Chicago, III). A P value <0.05 was considered to be statistically significant.

### Results

### 8.1 Composition 1 Exhibits No Cytotoxicity to Smooth Muscle Cells

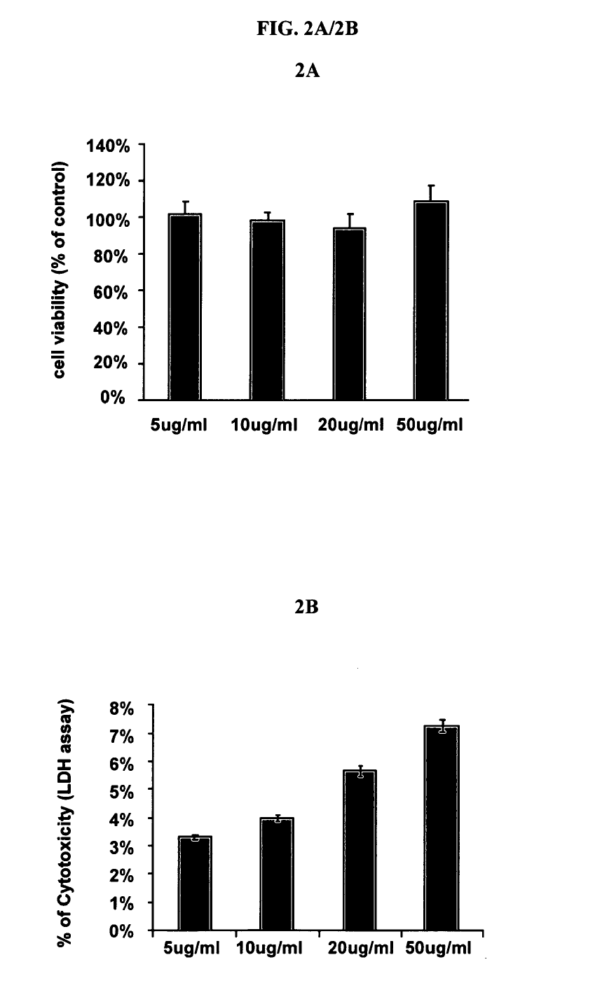

Potential cytotoxicity of Composition 1 to smooth muscle cells was tested. Composition 1 with different concentrations (ranged from 0 μm/ml-50 μm/ml) was individually added into A7r5 cell culture and incubated for 24 hours to examine survival rates of cells and cytotoxicity. As shown in FIG. 2A/2B, the cytotoxic effect of Composition 1 at different concentrations on smooth muscle cells (A7r5) was determined via MTT assay (FIG. 2A) and LDH assay (FIG. 2B). These results have shown that cell purification was not affected by the drug treatment and no cytotoxicity has been observed.

### 8.2 Composition 1 Effective Inhibits PDGF-Stimulated Smooth Muscle Cell Proliferation at Appropriate Concentrations

Effect of Composition 1 to smooth muscle cell (A7r5 cells) proliferation was investigated. Composition 1 with different concentrations (ranged from 5 μm/ml-50 μm/ml) was added into A7r5 cell culture. After incubating for 20 hours, platelet-derived growth factor (PDGF) was added and incubated for 24 hours to stimulate proliferation of smooth muscle cells. The effects of drugs on smooth muscle cell proliferation were observed by MTT assay and wound scratching test.

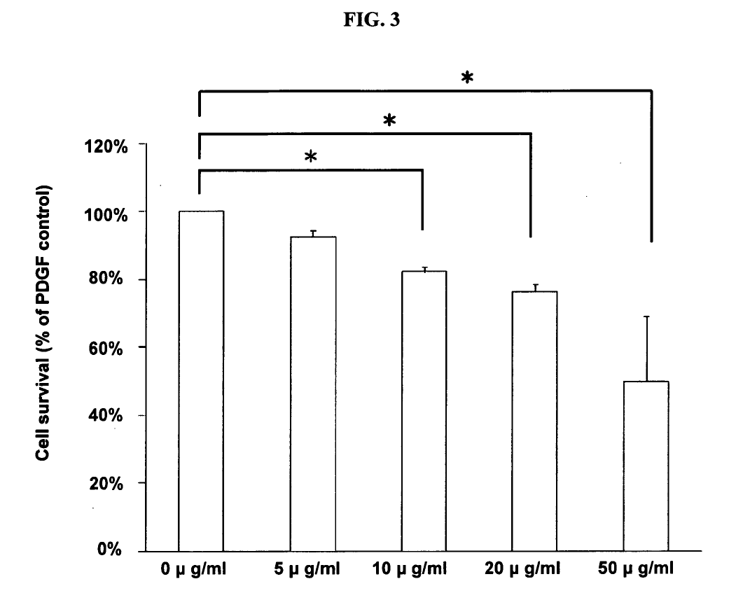

MTT assay result showed that Composition 1 has significantly inhibited PDGF-stimulated smooth muscle cell proliferation. As shown in FIG. 3, MTT assay result demonstrated that after incubation with PDGF for 24 hours, smooth muscle cell proliferation was effectively reduced by about 50% in treatment groups of Composition 1 (50 μm/ml).

### 8.3 Composition 1 Effective Inhibits PDGF-Stimulated Smooth Muscle Cell Migration at Appropriate Concentrations

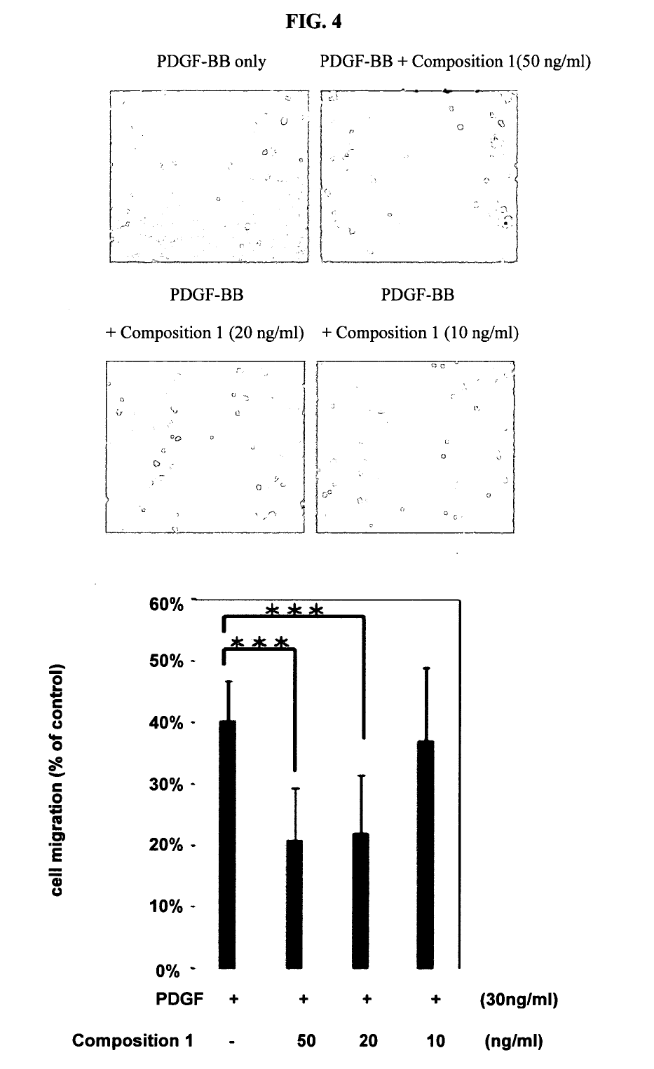

The inhibition of Composition 1 on migration of smooth muscle cells (A7r5 cells) was investigated in a wound scratching test by measuring PDGF-stimulated cell migration distance. The PDGF-stimulated cell culture without Composition 1 treatment was used as positive control. The result shows that the migration of the smooth muscle cells induced by PDGF was inhibited by Composition 1 in a dose-dependent manner. As shown in FIG. 4, treatment with Composition 1 (50 μm/ml) shows about 50% of decrease in smooth muscle cell migration.

8.4 Composition 1 Effectively Reduced Neointima Formation in Mice with Carotid Artery Ligation

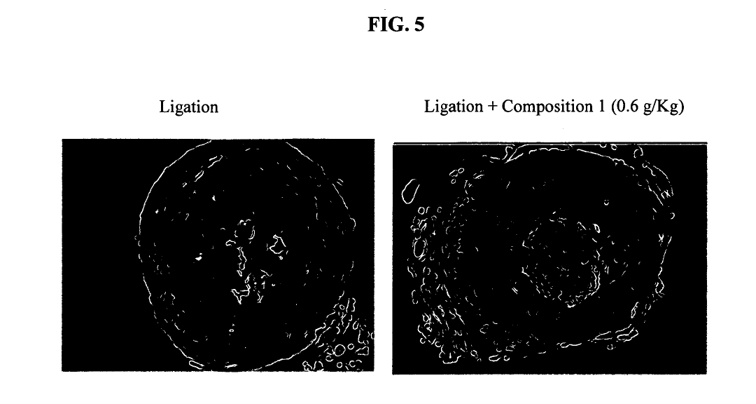

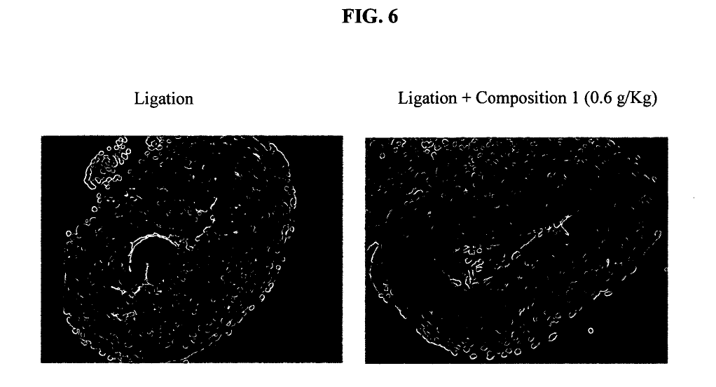

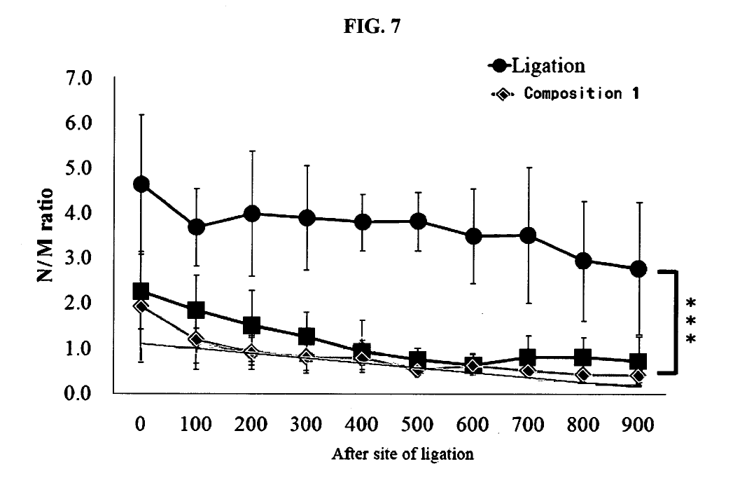

Three days prior to the operation, the mice were oral gavage fed with Composition 1 (0.6 kg/kg body weight), then the neointimal thickening was induced by carotid artery ligation. The mice were continuously treated for 28 days to study the effect of Composition 1 on neointima formation. In order to study the effect of the carotid artery ligation, Hematoxylin and eosin staining was performed to examine the thickening in media area and neointima area of carotid artery after ligation, as shown in FIG. 5 and FIG. 6, respectively. The treatment efficacy was evaluated based on lumen area, neointima area, media area and neotima/media ratio (N/M ration). As shown in FIG. 7, average N/M ratio was higher than 3.0 in control mice. However, average N/M ratio was lowered to 1.0 in mice treated with Composition 1. The reduction of neointima formation was statistically significant (p<0.001).

8.5 Treatment of Composition 1 in the Aortic Arch of Apo KO Mice Fed with High-Fat Diet

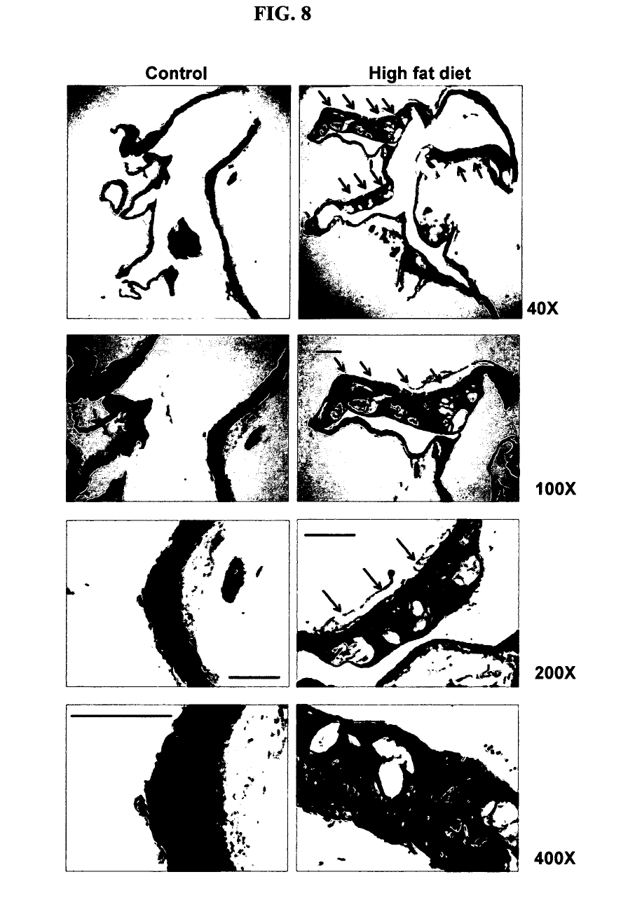

As shown in FIG. 8, fatty streaks and cholesterol deposition in aortic arch, foam cell formation, migration of smooth muscle cells and unstable fibrous plaques formation was observed in apoE-deficient mice (C57BL/6J background) fed with high fat diet. The amounts of Blood cholesterol, C reactive protein and ROS contents were measured in the apoE-deficient mice fed high-fat diet and gavaged with Composition 1 (0.6 kg/kg body weight).

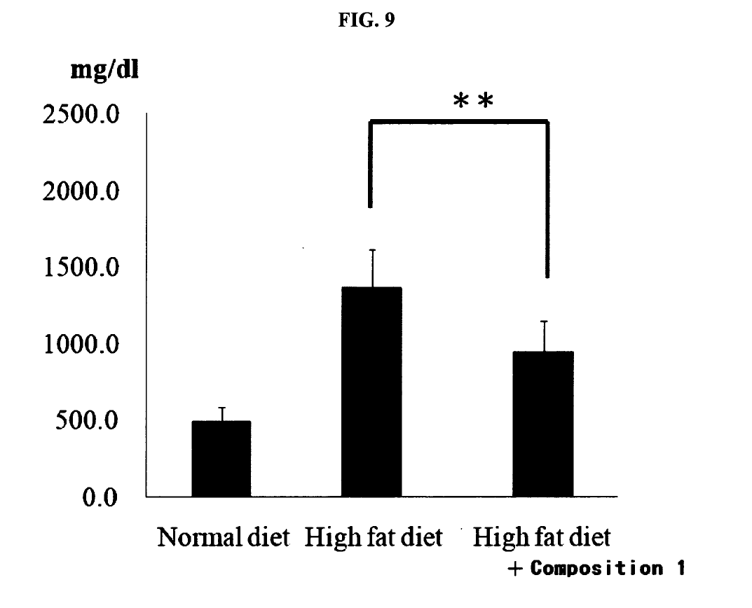

ApoE KO mice have undergone assessment of liver pathology. As shown in FIG. 9, the assessment of cholesterol concentration in blood showed that the reduction of cholesterol content by Composition 1 was statistically significant (p=0.002).

### Example 9

### Evaluation of the Efficacy and Safety of Composition 1 in Atherosclerosis Treatment

Primary outcome measures:

Change in neointima formation after 8 Weeks [Time Frame: Change from baseline and after 8 weeks of treatment]

### Secondary Outcome Measures:

Safety of Composition 1 in dose-escalation (adverse events and serious adverse events) is measured. Timeframe is one year.

### Criteria

Inclusion Criteria: subjects presenting type II or IIb primary hypercholesterolaemia diagnosed for at least 3 months, in a context of primary prevention with at least two associated cardiovascular risk factors and: (i) either “naive” to all lipid-lowering therapy, (ii) or treated with a statin (treatment ongoing or stopped during the previous 8 weeks).

### Arms

Composition 1: Experimental. Intervention: Drug: Composition 1.

### Assigned Intervention

Drug: Composition 1. Dosage form: 100 mg capsule bid×28 day cycles (Continuous treatment for a maximum of 1 year).

The results show that patients who take Composition 1 show reduction of neointima formation. The patients receiving Composition 1 have less atherosclerosis related symptoms or no symptoms. The invention compositions are therefore promising candidates for the treatment of atherosclerosis.

### Example 10

### Parenteral Formulation

To prepare a parenteral pharmaceutical composition suitable for administration by injection, 100 mg of a Composition described herein is dissolved in DMSO and then mixed with 10 mL of 0.9% sterile saline. The mixture is incorporated into a dosage unit form suitable for administration by injection.

### Example 11

### Oral Formulation

To prepare a pharmaceutical composition for oral delivery, 100 mg of an exemplary Composition 1 is mixed with 100 mg of corn oil. The mixture was incorporated into an oral dosage unit in a capsule, which is suitable for oral administration.

In some instances, 100 mg of Composition 1 described herein is mixed with 750 mg of starch. The mixture is incorporated into an oral dosage unit for, such as a hard gelatin capsule, which is suitable for oral administration.

### Example 12

### Sublingual (Hard Lozenge) Formulation

To prepare a pharmaceutical composition for buccal delivery, such as a hard lozenge, mix 100 mg of Composition 1 described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 13

### Inhalation Composition

To prepare a pharmaceutical composition for inhalation delivery, 20 mg of Composition 1 described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example 14

### Rectal Gel Formulation

To prepare a pharmaceutical composition for rectal delivery, 100 mg of Composition 1 described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 15

### Topical Gel Composition

To prepare a pharmaceutical topical gel composition, 100 mg of Composition 1 described herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A pharmaceutical composition comprising a component of at least one species from the genus Sargassum; a component of at least one species from the genus Lonicera; and a component of at least one species from the genus Cimicifuga.

2. The composition according to claim 1, wherein said composition inhibits PDGF-stimulated smooth muscle cell proliferation or migration.

3. The composition according to claim 1, wherein said composition reduced neointima formation.

4. The composition of claim 1, wherein the atherosclerosis is associated with coronary artery disease, aneurysm, arteriosclerosis, myocardial infarction, embolism, stroke, thrombosis, angina, vascular plaque inflammation, vascular plaque rupture, Kawasaki disease, calcification or inflammation.

5. The composition of claim 1, wherein said composition inhibits the production or progression of one or more atherosclerotic lesions within the vasculature of a subject.

6. The composition according to claim 5, wherein the vasculature comprises a cardiac artery.

7. The composition according to claim 6, wherein the vasculature comprises an aorta.

8. The composition of claim 1, wherein said composition prevents or treats an inflammation-related arteriosclerotic vascular disease in a subject.

9. The composition of claim 1, wherein said composition reduces cholesterol in a subject.

10. The composition of claim 1, wherein said composition is administered parenterally or intravenously.

11. The composition of claim 1, wherein said composition is administered by injection.

12. The composition of claim 1, wherein said composition is administered orally.

13. The composition of claim 1, wherein said subject is human

14. The composition of claim 1, wherein the Sargassum species is selected from the group consisting of Sargassum siliquastrum Ag, Sargassum pallidum Ag, and Sargassum fusiforme Setch.

15. The composition of claim 1, wherein the Lonicera species is selected from the group consisting of Lonicera japonica Thunb, Lonicera periclymenum, and Lonicera sempervirens.

16. The composition of claim 1, wherein the Cimicifuga species is selected from the group consisting of Cimicifuga foetida, L. var. intermedia Regel, Cimicifuga simplex, Cimicifuga heracleifolia, Kom, Cimicifuga dahurica (Turcz.) Maxim and Cimicifuga racemosa (L.) Nutt.

17. The composition of claim 1, wherein said composition comprises extracts of a component of at least one species from the genus Sargassum; a component of at least one species from the genus Lonicera; and a component of at least one species from the genus Cimicifuga.

18. The composition of claim 17, wherein the extracts are produced by extraction with an organic solvent or an aqueous solvent.

19. The composition of claim 1, wherein said composition comprises a component of Sargassum siliquastrum Ag, a component of Lonicera japonica Thunb, and a component of Cimicifuga foetida, L. var. intermedia Regel.

20. A method for the treatment of atherosclerosis comprising administering to a subject a therapeutically effective amount of a composition of claim 1.
